(19) United States
(12) Patent Application Publication
(10) Pub. No.: US 2024/0424038 A1
(43) Pub. Date: Dec. 26, 2024

(54) BLUE-GREEN ALGAE-CONTAINING TABLET

(71) Applicant: DIC Corporation, Tokyo (JP)

(72) Inventors: Yasuyuki Imai, Ichihara-shi (JP); Yurino Koseki, Ichihara-shi (JP)

(73) Assignee: DIC Corporation, Tokyo (JP)

(21) Appl. No.: 18/273,796

(22) PCT Filed: Feb. 10, 2022
(86) PCT No.: PCT/JP2022/005228

§ 371 Date: Jul. 24, 2023

(30) Foreign Application Priority Data
Feb. 16, 2021 (JP) 2021-022408

(51) Int. Cl. A61K 35/748; A61K 9/20; A61K 35/00

(52) U.S. Cl. CPC A61K 35/748; A61K 9/2013; A61K 2035/115

(57) ABSTRACT

When a tablet is formed from blue-green algae powder alone, there are problems such as a deteriorated tableting ratio due to breaking, cracking, and the like and collapse due to moisture absorption and the like. During the formation of a tablet, an excipient such as a sucrose fatty acid ester and dextrin, and a binder need to be added, but the tablet recently does not satisfy a preference of consumers who would like to avoid the additives. The inventors of the present invention have done intensive study to solve the problems, and as a result, found that a formulation with blue-green algae powder and α-linolenic acid improves the tableting ratio of a blue-green algae-containing tablet, and completed the present invention.

### TECHNICAL FIELD

The present invention relates to a blue-green algae-containing tablet having an improved tableting ratio.

### BACKGROUND ART

Blue-green algae, especially Spirulina blue-green algae are known to contain phycocyanin that exerts various bioactivities, and various types of preparations are conventionally formed.

As a blue-green algae-containing preparation, an algae-containing food containing Spirulinaand various types of organic acids in combination (PTL 1), a health food containing dried Spirulinapowder and peanut sprout (PTL 2), and the like, have been reported.

### CITATION LIST

### Patent Literature

PTL 1: Japanese Unexamined Patent Application Publication No. 2007-82444
PTL 2: Japanese Unexamined Patent Application Publication No. 2009-60865

### SUMMARY OF INVENTION

### Technical Problem

There is an attempt to form a tablet from blue-green algae powder alone, but the tablet cannot be formed, and a problem such as collapse due to moisture absorption or the like arises. During the formation of a tablet, an excipient such as a sucrose fatty acid ester and dextrin, and a binder need to be added. However, there is a recent problem in that the tablet does not satisfy a preference of consumers who would like to avoid the additives.

An object of the present invention is to provide a blue-green algae-containing tablet having an improved tableting ratio.

### Solution to Problem

The inventors of the present invention have done intensive study to achieve the object, and as a result, found that a formulation with blue-green algae powder and α-linolenic acid improves tableting properties and tableting ratio of a blue-green algae-containing tablet, and completed the present invention.

That is, the present invention includes the following aspects.
[1] A tableted food or beverage containing blue-green algae powder and α-linolenic acid in a content of 0.0035 parts by weight or more relative to 100 parts by weight of the blue-green algae powder.
[2] The food or beverage according to [1], wherein the α-linolenic acid is derived from a plant.
[3] The food or beverage according to [1] or [2], wherein the plant is at least one selected from soybean and sesame.
[4] The food or beverage according to any one of [1] to [3], wherein the food or beverage consists of the blue-green algae powder and the α-linolenic acid.

### Advantageous Effects of Invention

The present invention can provide a formulation for improving the tableting ratio of the blue-green algae-containing tablet.

### DESCRIPTION OF EMBODIMENTS

Hereinafter, a food or beverage of the present invention will be described in detail, but constituent elements described below are an example of an embodiment of the present invention, and the present invention is not limited to the contents.

### FOOD OR BEVERAGE

A food or beverage of the present invention includes blue-green algae powder and α-linolenic acid as essential ingredients.

The algae in the present invention are blue-green algae of the genus Spirulina, Arthrospira, Aphanizomenon, Fisherella, Anabaena, Nostoc, Synechocystis, Synechococcus, Tolypothrix, Aphanothece, Mastigoclaus, or Pleurocapsa, or the like. Among them, blue-green algae of the genus Spirulina and Arthrospira algae, which are produced in an industrial scale and confirmed to be safe, are preferred, and blue-green algae of the genus Spirulina are more preferred.

The blue-green algae powder may be blue-green algae obtained by drying raw blue-green algae, a dried product obtained by drying raw blue-green algae by an ordinary method, or a product obtained by further crushing dried blue-green algae into a desired particle size. As the dried blue-green algae powder, a commercially available product can be used.

A drying method in production of the blue-green algae powder of the present invention is not particularly limited as long as the effects of the present invention can be obtained, and an ordinary method can be used. For example, as the drying method, a method for drying raw blue-green algae, such as drying under heating, warm air drying, or freeze-drying can be used. In particular, drying under heating and freeze-drying are preferred, and freeze-drying is the most preferred since a moisture content in the blue-green algae is sufficiently removed and flavor and an effective ingredient are stably held.

In the production of the blue-green algae powder of the present invention, a pulverization treatment may be carried out as long as the effects of the present invention can be obtained, and blue-green algae can be pulverized by an ordinary method. A pulverization method may be carried out, for example, using a pulverizer such as a grinding impact pulverizer, a pin mill, a jet mill, a shear pulverizer, a masscolloider, a grinder, a mill stone, or a low temperature-freezing pulverizer, or other known pulverizers.

The blue-green algae powder of the present invention is not particularly limited as long as the effects of the present invention can be obtained. Dried blue-green algae may be used as they are, or a product obtained by drying and pulverizing blue-green algae in no particular order may be used. Drying and pulverization may be carried out simultaneously.

The average particle diameter of the blue-green algae powder of the present invention is not particularly limited as long as the effects of the present invention can be obtained. It is preferably within the range of 10 μm or more and 500 μm or less, more preferably within the range of 30 μm or more and 125 μm or less in terms of easy handling, and particularly preferably within the range of 40 μm or more and 95 μm or less in terms of more favorable tableting properties. In the present invention, as the average particle diameter, an average particle diameter measured by a laser analyzing and scattering method using a particle diameter distribution measurement device can be employed.

The origin of the α-linolenic acid of the present invention is not particularly limited as long as the α-linolenic acid is α-linolenic acid usually obtained. α-linolenic acid derived from a plant such as soybean, sesame, perilla, perilla frutescens, or linseed is preferred, and α-linolenic acid derived from soybean or sesame is particularly preferred. As the α-linolenic acid of the present invention, α-linolenic acid extracted and/or isolated from a raw material may be used, or α-linolenic acid contained in a variety of food product raw material as an ingredient may be used as it is without isolation.

In the present invention, the α-linolenic acid is contained preferably in a content of 0.0035 parts by weight or more, more preferably within the range of 0.0039 parts by weight or more and 0.02 parts by weight or less in terms of improving the stability of a tableted food or beverage, and particularly preferably within the range of 0.007 parts by weight or more and 0.016 parts by weight or less in terms of further improving flavor, relative to 100 parts by weight of the blue-green algae powder.

In the tableted food or beverage of the present invention, the total amount of the blue-green algae powder and the α-linolenic acid mixed is not particularly limited as long as an effect of improving tableting properties of the present invention is obtained. The total amount of the blue-green algae powder and the α-linolenic acid relative to the total amount of the tableted food or beverage is preferably 50% by weight or more, more preferably 80% by weight or more in terms of improving a tableting ratio, particularly preferably 90% by weight or more in terms of obtaining a distinguishing improvement effect of tableting ratio, and the most preferably 100% by weight in terms of obtaining suitable tableting properties without adding the influence of another raw material.

The tableted food or beverage of the present invention is not particularly limited as long as it is a food or beverage obtained by tableting and molding. Examples thereof include health foods, foods with functional claims, foods for specified health uses, dietary supplement, and supplement.

The tableted food or beverage of the present invention may contain an ingredient that is usually blended in a food or beverage as long as the effects of the present invention can be obtained. For example, the tableted food or beverage can be prepared by an ordinary method using additives such as an excipient, a binder, a disintegrant, a lubricant, a preservative, an antioxidant, a tonicity agent, a buffer, a coating agent, a taste masking agent, a solution adjuvant, a base, a dispersant, a stabilizer, and a colorant in appropriate combination.

Examples of the excipient include starch and derivatives thereof (dextrin, carboxymethyl starch, etc.), cellulose and derivatives thereof (methyl cellulose, hydroxypropylmethyl cellulose, etc.), saccharides (lactose, saccharose, glucose, trehalose, etc.), citric acid and salts thereof, malic acid and salts thereof, and ethylene diamine tetraacetate and salts thereof.

As the binder, starch or a derivative thereof (pregelatinized starch, dextrin, etc.), cellulose or a derivative thereof (ethylcellulose, carboxymethyl cellulose sodium, hydroxypropylmethyl cellulose, etc.), gum arabic, tragacanth, gelatin, saccharides (glucose, saccharose, etc.), ethanol, or the like can be used.

As the disintegrant, starch or a derivative thereof (carboxymethyl starch, hydroxypropyl starch, etc.), cellulose or a derivative thereof (carboxymethyl cellulose sodium, crystalline cellulose, hydroxypropylmethyl cellulose, etc.), a carbonic acid salt (calcium carbonate, calcium hydrogen carbonate, etc.), tragacanth, gelatin, agar, or the like can be used.

Examples of the lubricant include stearic acid, calcium stearate, magnesium stearate, talc, titanium oxide, calcium hydrogen phosphate, dried aluminum hydroxide gel, sucrose fatty acid esters, and edible oils and fats.

Examples of the preservative include a p-hydroxybenzoate ester, sulfurous acid salts (sodium sulfite, sodium pyrosulfite, etc.), phosphoric acid salts (sodium phosphate, calcium polyphosphate, sodium polyphosphate, sodium metaphosphate, etc.), dehydroacetic acid, sodium dehydroacetate, sorbic acid, glycerol, and saccharides.

Examples of the antioxidant include a sulfurous acid salt (sodium sulfite, sodium hydrogen sulfite, etc.), erythorbic acid, L-ascorbic acid, cysteine, thioglycerol, butylhydroxyanisol, dibutylhydroxytoluene, propyl gallate, ascorbic acid palmitate, and dl-α-tocopherol.

Examples of the tonicity agent include sodium chloride, sodium nitrate, potassium nitrate, dextrin, glycerol, and glucose.

Examples of the buffer include sodium carbonate, hydrochloric acid, boric acid, and phosphoric acid salts (sodium hydrogenphosphate).

Examples of the coating agent include cellulose derivatives (hydroxypropyl cellulose, cellulose acetate phthalate, hydroxypropylmethyl cellulose phthalate, etc.), shellac, polyvinyl pyrrolidone, polyvinyl pyridine (poly-2-vinyl pyridine, poly-2-vinyl-5-ethyl pyridine, etc.), polyvinyl acetyl diethyl aminoacetate, polyvinyl alcohol phthalate, and a methacrylate-methacrylic copolymer.

Examples of the taste masking agent include saccharides (glucose, saccharose, lactose, etc.), saccharin sodium, and sugar alcohols.

Examples of the solution adjuvant include ethylenediamine, nicotinic acid amide, saccharin sodium, citric acid, citric acid salts, sodium benzoate, polyvinyl pyrrolidone, polysorbate, sorbitan fatty acid esters, glycerol, propylene glycol, and benzyl alcohol.

Examples of the base include fats (lard, etc.), vegetable oil (olive oil, sesame oil, etc.), animal oil, lanolin acid, vaseline, paraffin, resin, bentonite, glycerol, and glycol oil.

Examples of the dispersant include gum arabic, tragacanth, a cellulose derivative (methyl cellulose etc.), sodium alginate, polysorbate, and sorbitan fatty acid esters.

Examples of the stabilizer include sulfurous acid salts (sodium hydrogen sulfite, etc.), nitrogen, and carbon dioxide.

Within the range that does not impair the effects of the present invention, the tableted food or beverage of the present invention can be prepared by an ordinary method using an optional food material, another active ingredient, or an additive acceptable for a food (e.g., a solvent, a softener, an oil, an emulsifier, an antiseptic, an acidulant, a sweetener, a bitter, a flavouring agent, a pH adjuster, a surfactant, a stabilizer, a colorant, an ultraviolet absorber, an antioxidant, a moisturizer, a thickener, a sticking agent, a dispersant, a flowability improver, a foaming agent, a wetting agent, a perfume, a flavoring material, a flavor modifier, etc.) in appropriate combination.

The active ingredient (biological active ingredient) or the like can be appropriately selected according to a purpose without particular limitation, and may be, for example, an ingredient such as amino acid, a saccharide, glycerol, vitamins such as vitamins A, B, C (ascorbic acid), D, and E, collagen, collagen peptide, hyaluronic acid, a hyaluronic acid salt, or ceramide.

A method for producing the tableted food or beverage of the present invention is not particularly limited as long as the effects of the present invention can be obtained. The tableted food or beverage is produced by a process including a mixing step of mixing various raw materials and a tableting step.

In the mixing step, mixing using a V-shaped mixer, a W-shaped mixer, or a Bohle container mixer can be employed. In particular, mixing using a V-shaped mixer is preferred.

In the tableting step, tableting using a rotary-type tablet press machine, a single-punch tablet press machine, or a double-punch tablet press machine can be employed. In particular, tableting using a rotary tablet press machine is preferred.

### EXAMPLES

Hereinafter, the present invention will be described in detail with reference to Examples, but the scope of the present invention is not limited to these Examples.

### BLUE-GREEN ALGAE POWDER

In the following Examples and Comparative Examples, Spirulinapowder available from DIC LIFETEC Co., Ltd., (dried Spirulinaalgae produced in an outdoor culture tank (spray dried product)) was used. The average particle diameter of the Spirulinapowder was 90 μm.

### PRODUCTION OF TABLET

### Examples 1 to 5 and Comparative Examples 1 to 4

In accordance with blending indicated in Table 1, powder raw materials were mixed to prepare about 3 kg of mixed powder. A specific production method was as follows. In mixing of each ingredient, the mixed powder obtained by mixing the powder raw materials was subjected to tableting and molding using a tablet press machine (HT-AP24SS-U, HATA TEKKOSHO Co., Ltd.) set at a rotational speed of 25 rpm, to produce a blue-green algae tablet (diameter: 8 mm, 200 mg).

For tableted products obtained in Examples 1 to 5, tableting properties, tablet thickness, hardness, and friability were evaluated and measured by the following methods.

### Evaluation of Tableting Properties

In the evaluation of tableting processing, visual evaluation was carried out in accordance with the following evaluation criteria (n=10).
O: tableting is possible. The appearance of all the tableted products is good.
Δ: tableting is possible. A product having an appearance failure such as tableting trouble, breaking, and cracking is obtained.
x: tableting is impossible.

### MEASUREMENT OF THICKNESS OF TABLET

### Measurement of Thickness and Hardness of Tablet

The hardness of the tablets was measured using a hardness tester (PTB311E Pharma Test Apparatebau). Each of the tablets was fixed in a sample holder of the tester, and measured. The thickness of 10 tablets in total was measured, and the average value was calculated.

### Friability Test

Abrasiveness and brittleness under impact against the tablets were tested using a tablet friability tester (TFT-120, Toyama Sangyo Co., Ltd.). Twenty tablets were used as samples in one drum, and the weight thereof before and after the test was measured. The rotational speed and the rotational time were set to 250 and 10 minutes, respectively. Twenty tablets were put in each of two drums, and the drums were rotated. After the rotation was completed, powder attached to the tablets was removed by air blowing, and the presence or absence of breaking and cracking were confirmed. Friability (%) was calculated by ((weight of tablet before test)−(weight of tablet after test))/(weight of tablet before test)×100.

### RESULTS

The results are indicated in Table 1.

TABLE 1

| | | | Content [g] | | | | | | |
|---|---|---|---|---|---|---|---|---|---|
| | | | of α-linolenic | | | Test items | | | |
| | | | acid relative | | | (n = 10, average) | | | |
| | Formulation | | to 100 g of | | | Tablet | | | Friability |
| | | Food raw | blue-green | Tablet | Tableting | thickness | Weight | Hardness | test |
| | Spirulina | material | algae powder | form | processing | [mm] | [mg] | [kgf] | [%] |
| Example 1 | 99.75% | Soybean powder | 0.0039 | Diameter: | ∘ | 4.18 | 200 | 3.7 | — |
| | | 0.25% | | 8, 200 mg | | | | | |
| Example 2 | 99.5% | Soybean powder | 0.0078 | Diameter: | ∘ | 4.22 | 201 | 4.1 | — |
| | | 0.5% | | 8, 200 mg | | | | | |
| Example 3 | 99% | Soybean powder | 0.0156 | Diameter: | ∘ | 4.26 | 198 | 3.2 | 0.16 |
| | | 1.0% | | 8, 200 mg | | | | | |
| Example 4 | 99% | Soybean powder | 0.0234 | Diameter: | Δ | 4.40 | 196 | 2.9 | 0.29 |
| | | 1.5% | | 8, 200 mg | | | | | |
| Example 5 | 97% | Soybean powder | 0.0468 | Diameter: | Δ | 4.33 | 194 | 2.2 | 0.14 |
| | | 3.0% | | 8, 200 mg | | | | | |
| Comparative | 100% | — | 0 | Diameter: | x | — | — | — | — |
| Example 1 | | | | 8, 200 mg | | | | | |
| Comparative | 100% | — | 0 | Diameter: | x | — | — | — | — |
| Example 2 | | | | 9, 200 mg | | | | | |
| Comparative | 98.5% | Sesame powder | 0.0030 | Diameter: | x | — | — | — | — |
| Example 3 | | 1.5% | | 8, 200 mg | | | | | |
| Comparative | 99.9% | Soybean powder | 0.00156 | Diameter: | x | — | — | — | — |
| Example 4 | | 0.1% | | 8, 200 mg | | | | | |

In Examples 1 to 5 in which the content of α-linolenic acid was 0.0035 parts by weight or more relative to 100 parts by weight of the blue-green algae powder, flowability was good, filling of a mortar of the tablet press machine was good, and tableting processing was smoothly carried out. In Comparative Examples 1 and 2 in which α-linolenic acid was not contained, tableting processing was impossible due to tableting trouble. In Comparative Examples 3 and 4 in which mixed powder in which the content of α-linolenic acid was low relative to 100 parts by weight of the blue-green algae powder was used, tableting trouble was caused.

As indicated in Examples, the tableting ratio of the blue-green algae powder was confirmed to be improved by a formulation of the present invention.

1. A tableted food or beverage comprising blue-green algae powder and α-linolenic acid in a content of 0.0035 parts by weight or more relative to 100 parts by weight of the blue-green algae powder.

2. The food or beverage according to claim 1, wherein the α-linolenic acid is derived from a plant.

3. The food or beverage according to claim 2, wherein the plant is at least one selected from soybean and sesame.

4. The food or beverage according to claim 1, wherein the food or beverage consists of the blue-green algae powder and the α-linolenic acid.

5. The food or beverage according to claim 2, wherein the food or beverage consists of the blue-green algae powder and the α-linolenic acid.

6. The food or beverage according to claim 3, wherein the food or beverage consists of the blue-green algae powder and the α-linolenic acid.
